(19) United States
(12) Patent Application Publication
(10) Pub. No.: US 2008/0077097 A1
(43) Pub. Date: Mar. 27, 2008

(54) Menstrual Cup

(75) Inventors: Carinne Chambers, Kitchener (CA); Francine Chambers, Kitchener (CA)

Correspondence Address: WOODCOCK WASHBURN LLP, CIRA CENTRE, 12TH FLOOR, 2929 ARCH STREET, PHILADELPHIA, PA, 19104-2891, US

(73) Assignee: Diva International Inc., Kitchener, ON (CA)

(21) Appl. No.: 11/663,470

(22) PCT Filed: Sep. 22, 2005
(86) PCT No.: PCT/CA05/01443

§ 371 Date: Mar. 22, 2007

(30) Foreign Application Priority Data
Sep. 23, 2004 (US) 60/612151

(51) Int. Cl. A61F 5/451

(52) U.S. Cl. 604/330

(57) ABSTRACT

A menstrual cup for use in a vagina. The cup includes a receptacle extending from an open top end to a closed bottom end and a stem attached to the receptacle at the bottom end thereof for use in removal of the cup. The receptacle comprises a wall having an inner wall surface defining a cavity adapted for collecting fluid and an opposed outer wall surface. The open top end has a predetermined diameter, the receptacle being adapted to be flexible and resilient. The outer wall surface includes an upper rim at the top end of the receptacle for strengthening the top end, a main portion extending from the bottom end of the receptacle portion towards the top end, and a transition portion between the main portion and the upper rim, the transition portion being streamlined to define a profile thereof which is substantially smooth.

### FIELD OF THE INVENTION

This invention is related to menstrual cups.

### BACKGROUND OF THE INVENTION

Menstrual cups are known, and various cups are disclosed in U.S. Pat. Nos. 1,891,761 (Goddard), 1,996,242 (Hagedom), 2,089,113 (Chalmers), Des. 323,212 (Crawford), and 5,827,248 (Crawford).

However, known menstrual cups have a number of disadvantages. For instance, a typical cup includes a relatively sharply defined ridge positioned generally in the vicinity of an upper end thereof, which tends to interfere with the removal of the cup. Although this relatively sharp ridge may assist in holding the cup in place, the ridge tends to interfere with removal of the cup, thereby sometimes causing discomfort. Also, known cups can include a plurality of holes disposed proximal to the ridge, to equalize air pressure inside and outside the cup when it is inserted and when it is removed, thereby facilitating insertion and removal. In addition, the known cups generally lack convenient and easily gripped portions at the bottom ends thereof, to facilitate removal. Finally, known cups are usually made of a thermoplastic such as polyethylene which, although suitably flexible and resilient, cannot be boiled for sterilization thereof. Alternatively, known cups may be made of natural latex rubber, however, this material has the disadvantage that many users are severely allergic to it.

Therefore, there is a need for an improved menstrual cup which overcomes one or more of the disadvantages of the prior art.

### SUMMARY OF THE INVENTION

In its broad aspect, the invention provides a menstrual cup for use in a vagina. The cup includes a receptacle extending from an open top end to a closed bottom end and a stem attached to the receptacle at the bottom end thereof, for use in removal of the cup. The receptacle includes a wall having an inner wall surface defining a cavity adapted for collecting fluid and an opposed outer wall surface. The open top end has a predetermined diameter, and the receptacle is adapted to be flexible and resilient. The wall comprises an upper rim portion at the top end of the receptacle for strengthening the top end, to maintain the cup in a preselected position in the vagina. Also, the wall comprises a transition portion extending-downwards from the rim portion towards the bottom end and a main portion extending from the transition portion to the bottom end of the receptacle portion. The transition portion is streamlined to define a profile which is substantially smooth.

In another aspect, the cup includes one or more ridges on the stein.

The invention additionally includes one or more receptacle ridges in the main portion of the outer wall surface.

In yet another aspect, the invention includes one or more volume markings on the inner wall surface, for indicating the volume of fluid collected in the cavity.

In yet another of its aspects, the invention additionally includes one or more holes, each hole extending from an inner end at the inner wall surface to an outer end at the outer wall surface to permit fluid communication between the inner wall surface and the outer wall surface when the cup is inserted in the vagina and removed therefrom, the inner end of each hole being positioned lower than the outer end.

In another aspect, the cup is made of silicone rubber.

### BRIEF DESCRIPTION OF THE DRAWINGS

The invention will be better understood with reference to the drawings, in which:

FIG. 1 is an isometric view of a preferred embodiment of a cup of the invention;

FIG. 2 is a side view of the cup of FIG. 1;

FIG. 3 is a top view of the cup of FIG. 1; and

FIG. 4 is a cross-section of the cup of FIG. 1.

### DETAILED DESCRIPTION OF THE PREFERRED EMBODIMENT(S)

Reference is first made to FIG. 4 to describe a preferred embodiment of a cup in accordance with the invention indicated generally by the numeral 20. The cup 20, which is adapted for use in a vagina (not shown), includes a receptacle 22 having a wall 25 with an inner wall surface 24 which defines a cavity 26 adapted for collecting fluid (not shown), and an opposed outer wall surface 28. The receptacle 22 extends from an open top end 30 to a closed bottom end 32. The top end 30 preferably has a predetermined-outside diameter (shown as “D” in FIG. 4) and the receptacle 22 is adapted to be flexible and resilient, as will be described. The wall 25 includes an upper rim portion 34 which strengthens the top end 30, to maintain the cup 20 in a pre-selected position (not shown) in the vagina. Also, the wall 25 includes a main portion 36 thereof extending from the bottom end 32 towards the top end 30. The wall 25 additionally includes a transition portion 38 between the main portion 36 and the upper rim portion 34 which is streamlined to define a profile thereof which is substantially smooth (FIG. 4).

The thickness of the cup wall 25, i.e. the distance between the inner wall surface 24 and the outer wall surface 28, varies from the upper rim portion 34 to the main portion 36, particularly in the transition portion 38 as best seen in FIG. 4. Generally, the thickness of the wall 25 decreases from the upper rim portion 34 to the transition portion 38 and from the transition portion 38 to the main portion 36. The upper rim portion 34 of the wall 25 is rounded at its top end and forms the thickest part of the wall 25 to strengthen the top, end 30 and to maintain both cup shape and position when in place in the vagina. The transition portion 38 of the wall tapers from the thicker upper rim portion 34 to the main portion 36 which is narrower. The transition portion 38, thus, provides additional strength to the cup to retain shape and position. The transition portion 38 also permits the cup to maintain a substantially smooth profile for increased comfort during use due to its tapered wall thickness as opposed to the defined ridge provided in many prior art cups. The thickness of the wall 25 at the narrow end of the transition portion 38 generally extends into and remains constant in the main portion 36. The main portion 36 need not necessarily comprise a constant wall thickness, however. The narrower wall thickness in the main portion 36 allows ready folding of the cup 20 for both insertion into and removal from the vagina. Although various wall thicknesses for the rim portion 34, transition portion 38 and main portion 36 will be appropriate, as one of skill in the art will appreciate, in one embodiment of the present invention, the wall thickness of the rim portion 34 is about 5 mm, the wall thickness of the transition portion 38 tapers from a thickness of about 3 mm to a thickness of about 2.2 mm and the thickness of the wall throughout the main portion 36 is maintained at about 2.2 mm.

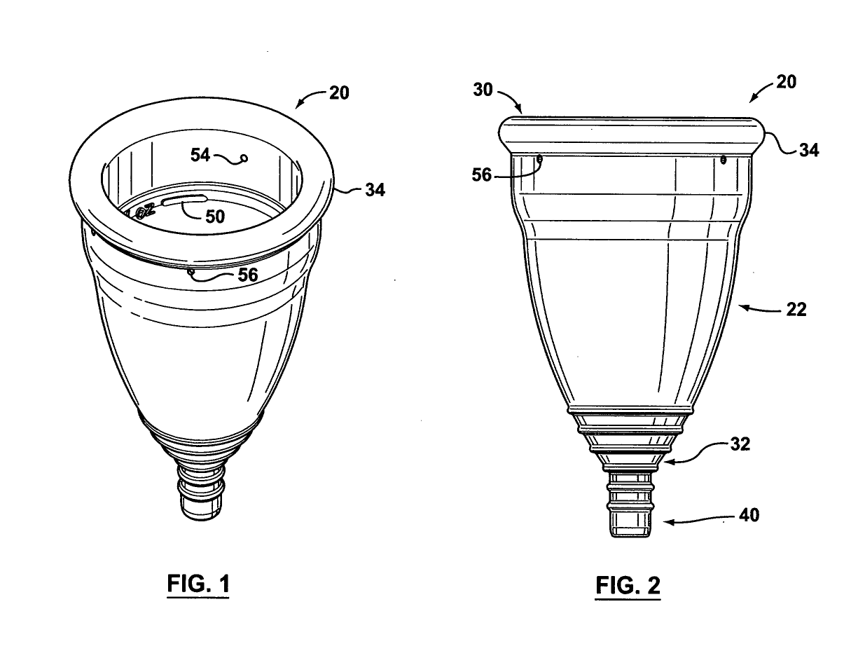

The cup 20 also includes a stem 40 which is attached to the receptacle 22 at the bottom end 32, for use in insertion and removal of the cup 20, as will be described. Preferably, the cup 20 includes one or more ridges 42 located on the stem 40 (FIGS. 1, 2). The ridges 42 make the stem 40 easier for a user (not shown) to grasp, thereby facilitating removal of the cup 20. Also, the preferred embodiment of the cup 20 additionally includes one or more receptacle ridges 44 in the outer surface 28 of the main portion 36, to make the cup 20 generally easier for the user to grasp and remove (FIGS. 1, 2).

As shown in FIGS. 1 and 4, in the preferred embodiment, the cup 20 includes one or more markings 50 on the receptacle 22, to enable the user to readily determine the volume of fluid accumulated therein. Preferably, the volume markings 50 are on the inner wall surface 24, and are provided in imperial units as well as in metric units for the user's convenience. The markings 50 are preferably positioned on the inner wall surface 24 so that the outer wall surface can be as smooth as possible for maximum comfort during use.

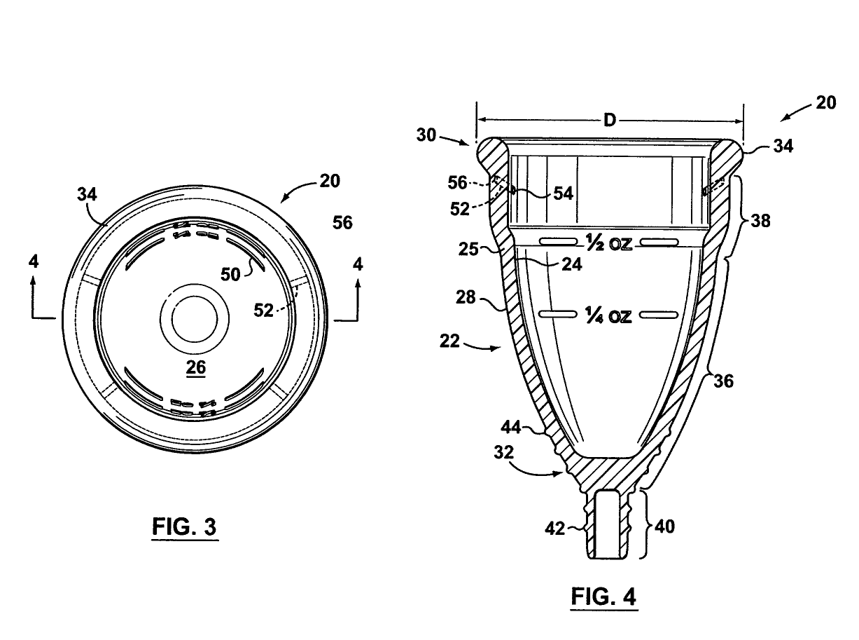

In the preferred embodiment, the cup 20 also includes one or more holes 52 (FIGS. 3, 4). Each hole 52 extends from an inner end 54 located at the inner wall surface 24 to an outer end 56 located at the outer wall surface 28. The holes 52 preferably are radially spaced apart from each other (FIG. 3). As shown in FIG. 4, when the cup 20 is substantially upright, the inner end 54 is positioned substantially lower than the outer end 56 of the hole 52. Because of the upwardly angled orientation of the holes 52, fluid which has accumulated in the cavity 26 is less likely to leak out through the holes 52, either when the cup 20 is in the vagina or as it is removed. Preferably, the holes 52 have a diameter of about 1 millimetre. The holes 52 permit fluid communication between the inner wall surface 24 and the outer wall surface 28 as the cup 20 is inserted into the vagina and removed therefrom, to equalize air pressure inside the cavity 26 with atmospheric air pressure, i.e., air pressure outside the outer wall surface 28. The outer ends 56 preferably are positioned at the intersection of the upper rim portion 34 and the transition portion 38, as will be described.

Preferably, the cup 20 comprises an elastomeric material, and the receptacle 22 and the stem 40 are integrally joined. The cup 20 is primarily formed in an injection mold, and it is therefore convenient, and expedient, to form the receptacle 22 and the stem 40 of the same material.

The preferred elastomeric material is an organosilicon oxide polymer, i.e., a silicone rubber. Silicone rubber is preferred because it rarely (if ever) causes skin irritation, and it has the necessary resiliency and durability. Also, silicone rubber can be placed in boiling water for sterilization without damaging the article. The silicone rubber is preferably a medical grade.

In use, the user folds the receptacle 22 lengthwise and inserts the cup 20 into the user's vagina, top end 30 first. Once inserted, the top end 30 returns to its usual size, i.e., the top end 30 returns to the predetermined diameter. The cup 20 preferably is positioned relatively low in the vagina so that the cup 20 may be easier to remove and to prevent leakage. When the user wishes to remove the cup 20, the user grasps the stem 40 and the bottom end 32, to pull the cup 20 out. The ridges 44 on the main portion 36 of the outer wall surface 28, and the ridges 42 on the stem 40, facilitate the removal of the cup 20 because the ridges 42, 44 make it easier for the user to grasp the cup 20. Because the stem 40 forms an integral unit with the receptacle 22, the stem 40 is not detachable. The stem 40 is preferably hollow, so that it is relatively flexible, and less likely to cause discomfort to the user when the cup 20 is inserted.

As shown in FIGS. 2 and 4, the upper ends 56 of the holes 52 are positioned in a recessed area where the upper rim portion 34 and the transition portion 38 meet. It is important that the upper ends 56 be in fluid communication with the surrounding environment when the user wishes to remove the cup 20. Otherwise, the air pressure inside and outside the cavity 26 would then not be equalized, and in those circumstances, removal of the cup 20 would result in suction directed to the vagina. The user folds the cup 20 lengthwise, to a limited extent, and this serves to ensure that at least some of the holes 52 permit air pressure equalization. Because of this, the outer ends 56 of the holes 52 are preferably positioned in the recessed area, so that the cavity 26 can remain substantially in fluid communication with the surrounding environment as the cup 20 is removed.

It therefore can be seen that the transition portion 38, having a streamlined profile, permits a relatively easy as well as comfortable removal of the cup 20, while the intersection of the transition portion 38 with the upper rim portion 34 provides a small recessed region in which the outer ends 56 of the holes 52 may desirably be located.

It will be appreciated by those skilled in the art that the invention can take many forms, and that such forms are within the scope of the invention as claimed. Therefore, the spirit and scope of the appended claims should not be limited to the descriptions of the preferred versions contained herein.

We claim:

1. A menstrual cup for use in a vagina, the cup comprising:
a receptacle comprising a wall having an inner wall surface defining a cavity adapted for collecting fluid and an opposed outer wall surface, the receptacle extending from an open top end to a closed bottom end, the open top end having a predetermined diameter, the receptacle being adapted to be flexible and resilient, the wall comprising an upper rim portion at the top end of the receptacle for strengthening the top end and to maintain the cup in a pre-selected position in the vagina, a transition portion extending downwards from the upper rim portion, the transition portion being streamlined to define a profile thereof which is substantially smooth, and a main portion extending downwards from the transition portion to the closed bottom end of the receptacle.

2. NA menstrual cup according to claim 1, wherein the thickness of the wall decreases from the upper rim portion to the transition portion and from the transition portion to the main portion.

3. A menstrual cup according to claim 1, wherein the thickness of the wall in the transition portion is tapered from the upper rim portion to the main portion.

4. A menstrual cup according to claim 1, additionally including a stern attached to the receptacle at the bottom end thereof, for use in removal of the cup.

5. A menstrual cup according to claim 4, additionally including at least one ridge on the stem.

6. A menstrual cup according to claim 1, additionally including at least one receptacle ridge in the main portion of the wall.

7. A menstrual cup according to claim 1 additionally including at least one volume marking on the inner wall surface, for indicating the volume of fluid collected in the cavity.

8. A menstrual cup according to claim 1, additionally including at least one volume marking on the outer wall surface, for indicating the volume of fluid collected in the cavity.

9. A menstrual cup according to claim 1, additionally including at least one hole extending from an inner end at the inner wall surface to an outer end at the outer wall surface to permit fluid communication between the inner wall surface and the outer wall surface as the cup is inserted in the vagina and removed therefrom, the inner end being positioned lower than the outer end.

10. A menstrual cup according to claim 1 comprising an elastomeric material.

11. A menstrual cup according to claim 10 in which the elastomeric material is a silicone rubber.
